(19) United States
(12) Patent Application Publication
(10) Pub. No.: US 2008/0213395 A1
(43) Pub. Date: Sep. 4, 2008

(54) Method for the Treatment of Gastrointestinal and Other Disorders with an Admixture of Vitamins

(75) Inventor: Mark Tuffley, Queensland (AU)

Correspondence Address: SUMMA, ALLAN & ADDITON, P.A., 11610 NORTH COMMUNITY HOUSE ROAD, SUITE 200, CHARLOTTE, NC, 28277, US

(73) Assignee: ADVENTURES PLUS PTY LTD, Brisbane (AU)

(21) Appl. No.: 11/577,114

(22) PCT Filed: Oct. 14, 2005
(86) PCT No.: PCT/AU05/01597

§ 371 Date: Nov. 27, 2007

(30) Foreign Application Priority Data
Oct. 14, 2004 (AU) 2004905959

(51) Int. Cl. A61K 33/00; A61K 33/06; A61K 33/30; A61K 33/32; A61K 33/34; A61K 33/24; A61K 33/04; A61P 1/00

(52) U.S. Cl. 424/638; 424/682; 424/722; 424/641; 424/639; 424/655; 424/702

(57) ABSTRACT

A method for the treatment and/or prophylaxis of disorders of the gastrointestinal system, other disorders selected from post exercise recovery, heat stress, vasovagal states, sleep disorders and fluid loss, and for the maintenance of blood sugar level homeostasis, comprising orally administering to said patient an admixture of vitamins and minerals formulated to have a pH greater than or equal to 5.0 in aqueous solution.

### TECHNICAL FIELD

The present invention relates to methods for the treatment and/or prophylaxis of disorders of the gastrointestinal system, disorders of the human body selected from the group consisting of post-exercise recovery, heat stress, vasovagal states, sleep disorders and fluid loss, and methods for the maintenance of blood sugar level homeostasis involving the administration of an admixture of vitamins and minerals. The invention relates particularly, but not exclusively, to mammals including human, canine and equine animals.

### BACKGROUND ART

Vitamins and minerals are necessary for the proper functioning of human and animal metabolism, and are present in food. However, nutritional supplementation is commonplace, and vitamin and mineral supplements of this type are well known.

These supplements are often in the form of tablets or capsules but are also available in effervescent form designed for dissolution in water. Such effervescent formulations typically have a very low pH, generally about between 2.8 and 4, as very low pH beverages are much more palatable than beverages with a higher pH. However, continued consumption of very low pH beverages may irritate the stomach and cause gastric problems. Acidic drinks can also exacerbate a condition known as metabolic acidosis found in people who perform regular, strenuous physical activity and other metabolic abnormalities such as renal failure. Furthermore, tooth erosion is a significant problem with repeated consumption of such beverages. In order to alleviate the disadvantages of such nutritional supplements, Australian patent no. 769792 proposes a beverage comprising vitamins and minerals in aqueous solution at a pH of 5.0 or above. This formulation comprises a fruit juice product, a vitamin and mineral supplement, a flavouring agent and water so that a palatable product is obtained despite the pH being greater than or equal to about 5.0. However, no therapeutic purpose is envisaged.

### SUMMARY OF THE INVENTION

The present invention relates to the use of an admixture of vitamins and minerals formulated so as to have a pH of greater than or equal to 5.0 in aqueous solution in the treatment or prophylaxis of disorders of the gastrointestinal system, certain functional disorders of the human body and in the control of hyperglycaemia. Thus, the present invention relates to the use of these supplements in therapy rather than in dietary supplementation.

According to a first aspect of the present invention there is provided a method for the treatment and/or prophylaxis of disorders of the gastrointestinal system in a patient in need of such treatment, comprising orally administering to said patient an admixture of vitamins and minerals formulated to have a pH greater than or equal to 5.0 in aqueous solution.

In an embodiment of the invention said admixture of vitamins and minerals is administered at a pH between 5.0 and 8.0, preferably between 7.0 and 8.0. Typically said admixture of vitamins and minerals is formulated in a composition which includes a buffer. While any appropriate buffer may be provided, typically the buffer comprises a sodium or potassium salt of an alkaline metal bicarbonate and/or carbonate and a carboxylic acid. The carboxylic acid may be selected from the group consisting of citric acid, tartaric acid, malic acid, succinic acid, adipic acid and fumaric acid, and is generally citric acid.

The term “vitamin” as used herein refers to an organic substance other than proteins, carbohydrates, and fats that is an essential constituent of the food of the animal. For the most part vitamins are substances that play an essential part in animal metabolic processes but which the animal cannot synthesise. However, certain animals can synthesise certain compounds of this group and all animals needing vitamin D can synthesise it in the presence of UV light. The vitamins are a well characterised group, and are generally named using letters of the alphabet. Specific examples are vitamin A, B group vitamin including vitamin B1 (thiamine), vitamin B2 (riboflavin), vitamin B3 (niacin), vitamin B12 (cyancobalamin), vitamin B6 (pyridoxine), folic acid, pantothenic acid, biotin, vitamin C (ascorbic acid), vitamin D, vitamin E and vitamin K.

As used herein the term “minerals” refers to trace elements required for normal metabolism. Minerals required for this purpose include sodium, magnesium, potassium, calcium, zinc, manganese, copper, selenium, and chromium.

In an embodiment the minerals in said admixture of vitamins and minerals comprise sodium, magnesium, potassium, calcium, zinc, manganese, copper, selenium and chromium.

In an embodiment, the vitamins in said admixture of vitamins and minerals comprises thiamine, riboflavin, niacin, vitamin C, vitamin D, vitamin E, vitamin B6, vitamin B12, pantothenate, biotin and folate.

The composition may comprise electrolytes additional to or in place of the minerals present in said admixture of vitamins and minerals. In particular, the electrolytes may include cations selected from calcium, magnesium, potassium and sodium and anions selected from chloride, phosphate, picolinate, sulfate and lactate.

In an embodiment said admixture further comprises a component selected from the group consisting of amino acids including essential amino acids, dietary fibre including soluble fibre, carbohydrates, bioflavonoids, fatty acids including essential fatty acids and flavouring agents.

The carbohydrate component may be a simple sugar. The sugar is preferably fructose, although may additionally or alternatively be dextrose, glucose, galactose, sucrose (in any of its forms including white sugar, raw sugar and brown sugar), or other sweeteners and is preferably about 0.75% wt in the administered form. However, the sugar may range from 0.5 g to 8.0 g per 100 ml in the administered form.

The essential fatty acids may comprise omega 3, omega 6 or omega 9 fatty acids as is present, for example, in fish oil.

Advantageously the admixture further comprises one or more amino acids selected from the group consisting of tryptophan, phenylalanine, arginine, glutamine, taurine and carnitine.

In an embodiment the composition further comprises insulin.

The term “gastrointestinal disorder” as used herein refers to a condition resulting from dysfunction in the gastrointestinal tract. A functional disorder of the gastrointestinal tract is one where the primary abnormality is an altered physiological function rather than an identifiable structure or biochemical cause. Irritable bowel syndrome and dyspepsia are among the most common functional gastrointestinal disorders. A gastrointestinal motility disorder is one in which movements of the digestive system, and the transit of the contents within it, are disrupted. This may occur when nerves or muscles in any portion of the digestive tract do not function in a strong coordinated fashion, whereupon a subject develops symptoms related to motility problems. These symptoms may range from heartburn to constipation. Other symptoms include abdominal distension, nausea, vomiting and diarrhoea.

As used herein the term “gastrointestinal motility disorders” refers to disorders in which the movements of the digestive system, and the transit of the contents within it, are disrupted. These conditions includes intestinal dysmotility, constipation, diarrhoea, gastroparesis and achalasia. Gastrointestinal motility disorders, nausea and vomiting, and gastroenteritis may be induced. For example, they may be induced by infection. They may also be induced by toxins including alcohol or chemotherapy or anaesthetics. Gastrointestinal motility disorders may also comprise gestational sickness, motion sickness, gastric erosion, colic (particularly equine and canine colic), and any nausea and vomiting associated with these conditions.

According to a second aspect of the present invention there is provided a method for the treatment and/or prophylaxis of a disorder of the human body selected from the group consisting of post-exercise recovery, heat stress, vasovagal states, sleep disorders and fluid loss, comprising orally administering to a patient in need of such treatment an admixture of vitamins and minerals formulated to have a pH greater than or equal to 5.0 in aqueous solution.

According to a third aspect of the present invention there is provided a composition comprising an admixture of vitamins and minerals, a buffer to allow delivery in aqueous solution at a pH of between 5.0 and 8.0, and insulin.

According to a fourth aspect of the present invention there is provided a method for the maintenance of blood sugar level homeostasis in a subject, comprising orally administering to said subject a composition comprising an admixture of vitamins and minerals formulated to have a pH greater than or equal to 5.0 in aqueous solution.

In an embodiment said subject is hyperglycaemic and administration of the composition results in a lowering of blood sugar levels.

In an embodiment said subject is hypoglycaemic and administration of the composition raises the blood sugar levels.

In an embodiment administration of the composition maintains blood sugar levels in a normal range.

Typically the composition is administered between 1 and 3 times following consumption of food to bring about a transition from a hyperglycaemic state to a normal range of blood sugar levels with minimal rebound to a hypoglycaemic state.

In an embodiment dosing occurs 3 times between completion of consumption and 1 hour thereafter.

In an embodiment dosing occurs at the time consumption finishes, then 10-15 minutes (preferably 12 minutes) and 30-35 minutes (preferably 32 minutes) thereafter. In an alternative embodiment dosing occurs 5-10 minutes after the time consumption finishes (preferably 6 minutes), then after 25-35 minutes (preferably after 26 minutes), then after 40-50 minutes (preferably 41 minutes).

In an embodiment said vitamin and mineral admixture is provided in powder form, but it may equally be in any other physical form for example, compressed into tablets or provided in the form of an aqueous solution.

In an embodiment said admixture of vitamins and minerals may be administered by mixing with a predetermined amount of flavouring such as fruit juice, as described in Australian Patent No. 769792, the contents of which are incorporated herein by reference. The predetermined amount of fruit juice is preferably up to about 30% by volume in the administered form. Said admixture of vitamins and minerals may also comprise a predetermined amount of fruit juice. The fruit juice may be derived from a nectar, or a concentrate which may be a substantially solid substance.

In an embodiment said admixture of vitamins and minerals may be administered by mixing into a pre-prepared food or beverage product and consuming said product. For example, a powder or tablet formulation may be added to food to effect the methods of treatment described above or for purposes of nutritional supplementation, with the advantage that such treatment/supplementation is achieved in a convenient and effective way with the advantages associated with reduced acidity described above.

Accordingly, in a fifth aspect of the present invention there is provided a method of administering an admixture of vitamins and minerals formulated to have a pH greater than or equal to 5.0 in aqueous solution to a subject, comprising:
(1) providing a food or beverage;
(2) providing an admixture of vitamins and minerals; and
(3) mixing the admixture of vitamins and minerals into the food or beverage and consuming same.

In an embodiment an admixture of vitamins and minerals formulated to have a pH greater than or equal to 5.0 in aqueous solution may be used in cooking as a replacement for sodium bicarbonate.

Accordingly, in a sixth aspect of the present invention there is provided a food product comprising an admixture of vitamins and minerals formulated to have a pH greater than or equal to 5.0 in aqueous solution, and food ingredients.

### MODES FOR PERFORMING THE INVENTION

In order that the nature of the present invention may be more clearly understood, preferred forms thereof will now be described with reference to the following non-limiting examples.

### EXAMPLE 1

One example of a vitamin and mineral admixture of the present invention is a powder formulation in which the powder comprises the following approximate quantities of the following substances for mixing with 375 ml of water and about 0.75% wt fructose:

| | | |
|---|---|---|
| Sodium | 127 | mg |
| Magnesium | 32 | mg |
| Potassium | 20 | mg |
| Calcium | 80 | mg |
| Zinc | 1.2 | mg |
| Manganese | 500 | μg |
| Copper | 0.15 | mg |
| Selenium | 7.0 | μg |
| Chromium | 10 | μg |
| Thiamine | 0.11 | mg |
| Riboflavin | 0.17 | mg |
| Niacin | 1.0 | mg |
| Vitamin C | 4.0 | mg |
| Vitamin D | 0.5 | μg |
| Vitamin E | 1.0 | mg |
| Vitamin B6 | 0.16 | mg |
| Vitamin B12 | 0.2 | μg |
| Pantothenate | 0.7 | mg |
| Biotin | 3.0 | μg |
| Folate | 20 | μg |
| Taurine | 6 | mg |
| Glutamine | 190 | mg |
| Carnitine | 10 | mg |
| Bicarbonate | 330 | mg |
| Chloride | 18 | mg. |

The pH of the resulting solution is about 7.4. In another form, the powder includes dextrose, either in place of or in addition to fructose. Fructose and dextrose comprise from 0.5 to 8 grams, per 100 mls. The powder can also include one or more amino acids, particularly one or more of the essential amino acids and dietary fibre.

The powder can also be mixed with up to about 30% vol fruit juice. The pH of the resulting solution ranges from about 5.0 to about 5.5. As an alternative to fructose, dextrose or fruit juice, the powder can be combined with, for example, honey and NaH(CO₃)₂ to give a resultant pH of about 8.0.

### EXAMPLE 2

Forty-five post general anaesthetic patients were orally administered a solution comprising the powder formulation of Example 1 and apple juice before consumption of any other drink or food. The results were recorded. The dosage and frequency of administration can be varied to suit the particular condition or situation. However, for best results it is recommended that small dosages are administered frequently. This was achieved by instructing patients to frequently sip the solution.

Solutions (as described above) were also administered to patients otherwise having conditions comprising or related to gastrointestinal motility disorders including associated pain, nausea and vomiting not related to gastrointestinal motility disorders and including associated pain, and gastroenteritis including associated pain; as well as conditions comprising or related to: other motility disorders; dyspepsia and heartburn; entero-colitis; post exercise recovery including testing under controlled conditions, while still under continuing load and also in the later part of an activity, and after heavy training blocks (eg VO2 max and/or anaerobic threshold); heat stress; vasovagal states; hyperglycaemic states; sleep disorders including those where circadian rhythm is disturbed; and fluid loss.

### Results

The solutions (described above) were rapidly absorbed from the upper gastrointestinal tract. None of the forty-five post general anaesthetic patients required post operative antiemetic injections. These patients also did not have any post operative vomiting and only one teenage female patient reported initially feeling a little nauseous and then settled fully, following consumption of the solution.

The solutions (described above) also otherwise prevented or improved conditions comprising or related to gastrointestinal disorders including associated pain, nausea and vomiting not related to gastrointestinal motility disorders and including associated pain, and gastroenteritis including associated pain; as well as conditions comprising or related to: other motility disorders; dyspepsia and heartburn; entero-colitis; post exercise recovery including testing under controlled conditions, while still under continuing load and particularly also in the later part of an activity, and particularly after heavy training blocks (eg VO2 max and/or anaerobic threshold); heat stress; vasovagal states; hyperglycaemic states; sleep disorders including those where circadian rhythm is disturbed; and fluid loss. Systemic body nourishment and hydration was rapidly achieved. The solution was also found to reverse upper gastrointestinal dysfunction and restore normal physiology.

### EXAMPLE 3

A further formulation, referred to herein as Base Jump/Calme 0.5 (BJ/C or BJ/C.5) is formulated as follows:

| Bicarbonate | | 62.5 | mg/gm |
|---|---|---|---|
| Omega 3 Fatty Acid | | 0.23 | mg/gm |
| Carnitine | | 2.0 | mg/gm |
| Glutamine | | 36.5 | mg/gm |
| Taurine | | 1.2 | mg/gm |
| Tryptophan | | 0.67 | mg/gm |
| Phenylalanine | | 1.8 | mg/gm |
| Arginine | | 5.5 | mg/gm |
| Vitamin E | | 0.2 | mg/gm |
| Vitamin D | | 0.9 | μg/gm |
| Vitamin C | | 0.8 | mg/gm |
| Vitamin B1 Thiamine | | 0.024 | mg/gm |
| Vitamin B2 Riboflavin | | 0.035 | mg/gm |
| Vitamin B3 Niacin | | 0.2 | mg/gm |
| Vitamin B6 | | 0.03 | mg/gm |
| Vitamin B12 | | 0.04 | μg/gm |
| Vitamin B5 Pantothenate | | 0.13 | mg/gm |
| Biotin | | 0.6 | μg/gm |
| Folate | | 4.0 | μg/gm |
| Zinc | | 0.24 | mg/gm |
| Magnesium | | 5.9 | mg/gm |
| Manganese | | 100.0 | μg/gm |
| Selenium | | 1.4 | μg/gm |
| Copper | | 0.027 | mg/gm |
| Chloride | | 3.5 | mg/gm |
| Chromium | | 2.0 | μg/gm |
| Calcium | | 16.0 | mg/gm |
| Protein | | 0.04 | mg/gm |
| Sugar | fructose | 0.185 | mg/gm |
| | dextrose | 0.185 | mg/gm |

| | | |
|---|---|---|
| Fibre (Inulin) | 0.2 | mg/gm |
| Sodium | 24 | mg/gm |
| Potassium | 3.9 | mg/gm |

### EXAMPLE 4

### Blood Glucose Response to Standardised Carbotest*

Test 1 - 50 gm Glucose/Same Subject

| | Standard | SC50 + 5 gm BJ/C0.5 |
|---|---|---|
| | Carbotest* 50 gm | in 300 mls water at |
| Blood Sugar Level | glucose | 0, 12, 32 minutes |
| Time Minutes | (SC 50) | i.e. 3 doses BJ/C.5 |
| 0 | 5.2 mmol/litre | 5.2 mmol/Litre |
| 2 | 5.6 | 5.0 |
| 4 | 5.6 | 5.2 |
| 6 | 5.4 | 5.7 |
| 8 | 6.1 | 6.0 |
| 10 | 6.3 | 6.8 |
| 15 | 7.4 | 6.6 |
| 30 | 8.4 | 6.8 |
| 45 | 8.1 | 6.4 |
| 60 | 7.1 | 5.1 |
| 90 | 5.4 | 5.1 |
| 120 | 3.7 | |

*Heritage Diagnostics, Sydney

This result is greater than 50% reduction in Glycaemic index (area under a graph showing elevated blood sugar levels).

Test 2 - 5 gm BJ/C in 300 mls of water was administered
to the subject at 6 minutes, 26 minutes and 41 minutes
after consumption of a high glycaemic load food product.

| Time/Minutes | Blood Sugar Level mml/l |
|---|---|
| 0 | 8.8 |
| 5 | 9.0 |
| 15 | 8.1 |
| 25 | 8.0 |
| 40 | 7.4 |
| 50 | 6.0 |
| 60 | 6.3* BSL stabilised |

The notable features of the tests are:
1. the subject felt unwell during the standard carbotest, flushed, more difficult cognition, needing a rest at the 45 minute mark from normal domestic duties but in the second test when BJ/C 0.5 was administered felt well, energetic and had clear thoughts.
2. There was virtually no overshoot or dipping below median/initial blood glucose level. This is a consistent finding in a hyperglycaemic induced state otherwise.
   Note: In all tests no other food or drink were consumed.
   Note: No overshoot although the blood sugar level stabilized at 6.0 to 6.3 mml/litre in the second Test—higher than the normal resting/fasting blood sugar level of about 5.5/5.4 mmol/litre).

### EXAMPLE 5

The formulation of Example 3 is an effective replacement for sodium bicarbonate in cooking and baking it enhances taste and improves consistency of the mixture so that it allows reduction of sugar (typically by 25%) and fat/binding agent (up to ⅓).

Each measure of bicarbonate is replaced by two to three measures of BJ/C.5.

The product rises more with BJ/C i.e. there is improved leavening and the product has a pleasing taste and consistency.

Further, it has a reduced glycaemic index of some 10-20% (depending upon the rate of BJ/C.5 additions and sugar/fat levels) but almost completely eliminates blood sugar level (BSL) dipping and rebound hypoglycaemia which can otherwise approximate the same magnitude of discrepancy from mean BSL negatively compared to BSL elevation.

A typical example is Anzac biscuits—the data below compares the effect of an original recipe and one containing BJ/C.5 as leavening and taste enhancing agent.

| | | Modified Recipe |
|---|---|---|
| | | BJ/C.5 |
| | | leavening |
| | | (with 25% less |
| BSL | | sugar + olive |
| Mmol/litre | Original Recipe | oil substitute |
| Time/Minutes | Bicarbonate leavening | for butter). |
| 0 | 5.4 | 4.9 |
| 10 | 5.5 | 5.8 |
| 20 | 6.0 | 6.1 |
| 30 | 6.1 | 5.2 |
| 40 | 7.1 | 5.0 |
| 50 | 5.9 | 5.2 BSL stabilised |
| 60 | 4.8 | |
| 70 | 4.1 | |
| 80 | 3.8 | |
| 90 | 3.8 | |
| 100 | 4.2 - still not returned | |
| | to mean BSL | |

The data demonstrated that the disturbance to BSL following consumption of the Anzac biscuits (both elevation and depression) lasts more than twice as long in the original recipe as in the food containing BJ/C.5 as a leavening taste/consistency enhancer. This is also consistent with the result seen with the standardised Carbotest 50 gm glucose, where BSL at 120 minutes (standard glycaemic index (G.1) testing time) still had not started towards returning to a mean level.

### In Summary

It is clear from these results that BJ/C.5 in a standard Carbotest 50 gm glucose and when combined with food.

1. is more rapidly absorbed i.e. nutrients.

2. has a reduced maximum blood sugar (glucose) level.

3. has a reduced glycaemic index (area under the positive/elevated position of the BSL response).

4. has a powerful buffering effect upon rebounding hypoglycaemia.

5. reduces the time of BSL disturbance from the individual's mean BSL zone by more than 50% *(under the above test conditions).

6. because of G.I. lowering properties the glycaemic load (G.L.) of foods containing BJ/C.5 is also lowered.

$$G . L . = \frac{G . I . \times amount of carbohydrate ( gms ) }{100}$$

Glycaemic load is a reliable predictor of the effect of a food on BSL.

Whilst not wishing to be bound by theory, it is believed that the formulations of the invention provide enhanced transport of nutrients across the cellular membranes of the gut and then the cellular membranes of bodily tissues including the brain and muscles. There is a clear implication of insulin sparing (reduced insulin response), facilitated glucose uptake by cells together with associated osmotic solute drag of water (flow) and cellular utilization of a wide range of nutrients/micronutrients included in BJ/C.5. These are powerful and highly advantageous properties consistent with health and well-being

### EXAMPLE 6

Base Jump/Calme (Example 3) when consumed in 5 gm/300 ml water has a mild initial lowering effect upon BSL when food has not been consumed.

Fasting BSL 5.5 mml/l

At 1 min—5 gm BJ/C in 300 mls H2O

10 min—5.0 mmol/l

At 11 min repeated—5 gm BJ/C in 300 mls H2O

20 min 5.3 mmol/l

60 min 5.4 mmol/l

While not wishing to be bound by theory, it is believed that the mild lowering of the BSL is due to enhanced cellular uptake of glucose with rapid reestablishment of homeostasis/normalisation of BSL even with a repeated dose of 5 gm BJ/C in 300 mls of water at 11 minutes. The implication of this is there may be a cellular update/membrane transport rate limiting step in part modulated and/or facilitated by the components of BJ/C and BJ/C.5.

Further the brain's only source of energy is glucose except in starvation) and approximates 50% of the obligatory energy requirements of the body. Disturbed blood glucose levels and impaired glucose transport eg in diabetes is known to adversely affect cognition. BJ/C.5 appears to be a powerful beneficial facilitator of this process.

This facilitation also seems to occur with strenuous muscle activity delaying the onset of lactic acidosis and is therefore beneficial to athletic performance.

In the claims which follow and in the preceding description of the invention, except where the context requires otherwise due to express language or necessary implication, the word “comprise” or variations such as “comprises” or “comprising” is used in an inclusive sense, ie. to specify the presence of the stated features but not to preclude the presence or addition of further features in various embodiments of the invention.

It is to be clearly understood that although prior art publication(s) are referred to herein, this reference does not constitute an admission that any of these documents forms part of the common general knowledge in the art in Australia or in any other country.

1-52. (canceled)

53. A method for the treatment of nausea and/or vomiting in a patient in need of such treatment, comprising orally administering to said patient a composition comprising at least one vitamin selected from the group consisting of thiamine, riboflavin, niacin, vitamin C, vitamin D, vitamin E, vitamin B, vitamin B12, pantothenate, biotin and folate, at least one mineral selected from the group consisting of sodium, magnesium, potassium, calcium, zinc, manganese, copper, selenium and chromium and at least one amino acid selected from the group consisting of glutamine, tryptophan, phenylalanine, arginine and carnitine, and a buffer formulated to maintain a pH greater than or equal to 5.0 in aqueous solution.

54. A method as claimed in claim 53 wherein said composition is formulated to have a pH between 5.0 and 8.0.

55. A method as claimed in claim 54 wherein said composition is formulated to have a pH between 7.0 and 8.0.

56. A method as claimed in claim 55 wherein said composition is buffered through addition of a sodium or potassium salt of an alkaline metal bicarbonate and/or carbonate and, optionally, a carboxylic acid.

57. A method as claimed in any one of claims 53 to 56 wherein said composition further comprises electrolytes additional to the minerals in said admixture of vitamins and minerals.

58. A method as claimed in any one of claims 53 to 57 wherein said composition further comprises one or more components selected from the group consisting of dietary fibre, soluble fibre, carbohydrates, bioflavonoids, fatty acids and flavouring agent.

59. A method as claimed in claim 58 wherein the carbohydrate is a simple sugar selected from the group consisting of dextrose, fructose, glucose, galactose and sucrose.

60. A method as claimed in claim 58 wherein the fatty acid is selected from the group consisting of omega 3, omega 6 and omega 9 fatty acids.

61. A method as claimed in claim 58 further comprising insulin.

62. A method as claimed in any one of claims 53 to 61 wherein said nausea and vomiting is related to a gastrointestinal motility disorder.

63. A method as claimed in claim 62 wherein said gastrointestinal motility disorder is selected from the group consisting of intestinal dysmotility, esophagitis, achalasia, gastritis, gestational sickness and motion sickness.

64. A method as claimed in any one of claims 53 to 61 wherein said nausea or vomiting is induced by infection or a toxin.

65. A method as claimed in claim 64 wherein said toxin is alcohol, a chemotherapeutic drug or an anaesthetic.
